(19) United States
(12) Patent Application Publication
(10) Pub. No.: US 2004/0097569 A1
(43) Pub. Date: May 20, 2004

(54) Aryl substituted hydantoin compounds and their use as sodium channel blockers

(75) Inventors: Qun Sun, Princeton, NJ (US); Donald J. Kyle, Newton, PA (US)

Correspondence Address: STERNE, KESSLER, GOLDSTEIN & FOX PLLC, 1100 NEW YORK AVENUE, N.W., WASHINGTON, DC, 20005, US

(73) Assignee: Euro-Celtique S.A.

(21) Appl. No.: 10/630,814

(22) Filed: Jul. 31, 2003

Related U.S. Application Data
(60) Provisional application No. 60/399,435, filed on Jul. 31, 2002.

(51) Int. Cl. A61K 31/4164; C07D 233/72

(52) U.S. Cl. 514/389; 548/317.1

(57) ABSTRACT

This invention relates to aryl substituted hydantoins of Formula I:

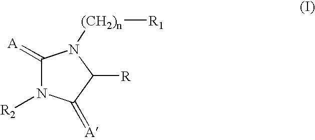

or a pharmaceutically acceptable salt, prodrug or solvate thereof, wherein n, A, A′, R, R₁ and R₂, are defined in the specification. The invention is also directed to the use of compounds of Formula I for the treatment of neuronal damage following global and focal ischemia, for the treatment or prevention of neurodegenerative conditions such as amyotrophic lateral sclerosis (ALS), and for the treatment, prevention or amelioration of both acute or chronic pain, as antitinnitus agents, as anticonvulsants, and as antimanic depressants, as local anesthetics, as antiarrhythmics and for the treatment or prevention of diabetic neuropathy.

### CROSS-REFERENCE TO RELATED APPLICATIONS

This application claims the benefit under 35 U.S.C. § 119(e) of U.S. Provisional Application No. 60/399,435, filed Jul. 31, 2002.

### BACKGROUND OF THE INVENTION

1. Field of the Invention

This invention is in the field of medicinal chemistry. In particular, the invention relates to novel aryl substituted hydantoins, and the discovery that these compounds are blockers of sodium (Na+) channels.

2. Related Art

Several classes of therapeutically useful drugs, including local anesthetics such as lidocaine and bupivacaine, antiarrhythmics such as propafenone and amioclarone, and anticonvulsants such as lamotrigine, phenytoin and carbamazepine, have been shown to share a common mechanism of action by blocking or modulating Na⁺ channel activity (Catterall, W. A., Trends Pharmacol. Sci. 8:57-65 (1987)). Each of these agents is believed to act by interfering with the rapid influx of Na⁺ ions.

Recently, other Na⁺ channel blockers such as BW619C89 and lifarizine have been shown to be neuroprotective in animal models of global and focal ischemia and are presently in clinical trials (Graham et al., J. Pharmacol. Exp. Ther. 269:854-859 (1994); Brown et al., British J. Pharmacol. 115:1425-1432 (1995)).

The neuroprotective activity of Na⁺ channel blockers is due to their effectiveness in decreasing extracellular glutamate concentration during ischemia by inhibiting the release of this excitotoxic amino acid neurotransmitter. Studies have shown that unlike glutamate receptor antagonists, Na⁺ channel blockers prevent hypoxic damage to mammalian white matter (Stys et al., J. Neurosci. 12:430-439 (1992)). Thus, they may offer advantages for treating certain types of strokes or neuronal trauma where damage to white matter tracts is prominent.

Another example of clinical use of a Na⁺ channel blocker is riluzole. This drug has been shown to prolong survival in a subset of patients with ALS (Bensimm et al., New Engl. J. Med. 330:585-591 (1994)) and has subsequently been approved by the FDA for the treatment of ALS. In addition to the above-mentioned clinical uses, carbamazepine, lidocaine and phenytoin are occasionally used to treat neuropathic pain, such as from trigeminal neurologia, diabetic neuropathy and other forms of nerve damage (Taylor and Meldrum, Trends Pharmacol. Sci. 16:309-316 (1995)), and carbamazepine and lamotrigine have been used for the treatment of manic depression (Denicott et al., J. Clin. Psychiatry 55:70-76 (1994)). Furthermore, based on a number of similarities between chronic pain and tinnitus, (Moller, A. R. Am. J. Otol. 18:577-585 (1997); Tonndorf, J. Hear. Res. 28:271-275 (1987)) it has been proposed that tinnitus should be viewed as a form of chronic pain sensation (Simpson, J. J. and Davies, E. W. Tips. 20:12-18 (1999)). Indeed, lignocaine and carbamazepine have been shown to be efficacious in treating tinnitus (Majumdar, B. et al. Clin. Otolaryngol. 8:175-180 (1983); Donaldson, I. Laryngol. Otol. 95:947-951 (1981)).

It has been established that there are at least five to six sites on the voltage-sensitive Na⁺ channels which bind neurotoxins specifically (Catterall, W. A., Science 242:50-61 (1988)). Studies have further revealed that therapeutic antiarrhythmics, anticonvulsants and local anesthetics whose actions are mediated by Na⁺ channels, exert their action by interacting with the intracellular side of the Na⁺ channel and allosterically inhibiting interaction with neurotoxin receptor site 2 (Catterall, W. A., Ann. Rev. Pharmacol. Toxicol. 10:15-43 (1980)).

A need exists in the art for novel compounds that are potent blockers of sodium channels, and are therefore useful for treating a variety of central nervous system conditions, including pain.

### SUMMARY OF THE INVENTION

One aspect of the present invention is directed to the novel aryl substituted hydantoins of Formula I.

The present invention is also related to the discovery that aryl substituted hydantoins represented by Formula I act as blockers of sodium (Na⁺) channels.

Another aspect of the present invention is directed to the use of novel compounds of Formula I as blockers of sodium channels.

The invention is also related with treating a disorder responsive to the blockade of sodium channels in a mammal suffering from excess activity of said channels by administering an effective amount of a compound of Formula I as described herein.

A further aspect of the present invention is to provide a method for treating, preventing or ameliorating neuronal loss following global and focal ischemia; treating, preventing or ameliorating pain including acute and chronic pain, and neuropathic pain; treating, preventing or ameliorating convulsion and neurodegenerative conditions; treating, preventing or ameliorating manic depression; using as local anesthetics and anti-arrhythmics, and treating tinnitus by administering a compound of Formula I to a mammal in need of such treatment or use.

Also, an aspect of the present invention is to provide a pharmaceutical composition useful for treating disorders responsive to the blockade of sodium ion channels, containing an effective amount of a compound of Formula I in a mixture with one or more pharmaceutically acceptable carriers or diluents.

Additional embodiments and advantages of the invention will be set forth in part in the description that follows, and in part will be obvious from the description, or can be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DETAILED DESCRIPTION OF THE INVENTION

Novel compounds of the present invention are aryl substituted hydantoins represented by Formula I:

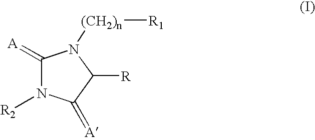

or a pharmaceutically acceptable salt, or solvate thereof, wherein:

n is 0 to 3;

A and A′ are independently oxygen or sulfur;

R is hydrogen, linear or branched C₁₋₆ alkyl, optionally substituted benzyl, thio(C₁₋₆)alkyl, C₁₋₆ alkylthio(C₁₋₆)alkyl, hydroxy(C₁₋₆)alkyl, amino(C₁₋₆)alkyl, guanidinyl(C₁₋₆)alkyl, carboxy(C₁₋₆)alkyl or aminocarboxy(C₁₋₆)alkyl;

R₁ is selected from the group consisting of:

(i) optionally substituted phenoxyphenyl;

(ii) optionally substituted benzyloxyphenyl;

(iii) optionally substituted phenylthiophenyl;

(iv) optionally substituted benzylthiophenyl;

(v) optionally substituted phenylaminophenyl;

(vi) optionally substituted benzylaminophenyl; and

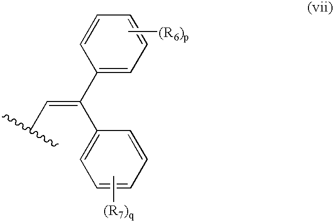

wherein each occurrence of R₆ and each occurrence of R₇ are independently C₁₋₆ alkyl, C₁₋₆ haloalkyl, C₁₋₆ alkoxy, C₁₋₆ hydroxyalkyl or C₁₋₆ alkoxy(C₁₋₆)alkyl; and

p and q are independently integers from zero to 4;

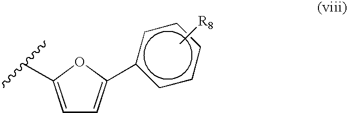

wherein R₈ is hydrogen, halogen, hydroxy, C₁₋₆ alkyl, C₁₋₆ alkoxy, cyano, amino, or nitro;

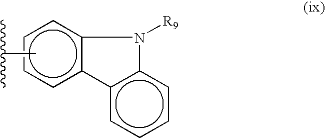

wherein R₉ is hydrogen or C₁₋₆ alkyl; and

(x) optionally substituted naphthyl; and

R₂ is selected from the group consisting of:

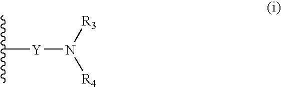

where

Y is an optionally substituted C₂₋₆ alkylene, and

R₃ and R₄ are the same or different and are hydrogen, alkyl, or aryl, or R₃ and R₄ together form an alkylene chain having 3 to 5 carbon atoms, optionally substituted with an alkyl or aryl moiety, or said alkylene chain is optionally interrupted by an oxygen atom or —NR₅, where R₅ is hydrogen or C₁₋₆ alkyl;

(ii) pyridylalkyl; and

(iii) piperidin-4-ylalkyl, optionally substituted by alkyl, aryl or aralkyl.

Preferred compounds of Formula I are those wherein R₁ is phenoxyphenyl or benzyloxyphenyl, wherein the phenyl group of the phenoxy or benzyloxy moiety is optionally substituted with alkyl, alkoxy, halogen or haloalkyl. Preferred substituents include one to three, preferably one or two, substituents independently selected from the group consisting of C₁₋₄ alkyl, C₁₋₄ alkoxy, halogen, or C₁₋₄ haloalkyl. Suitable values of R₁ in this embodiment of the invention include (3-phenoxy)phenyl, (4-phenoxy)phenyl, (3-benzyloxy)phenyl, (4-benzyloxy)phenyl, any of which is optionally substituted by one, two or three groups independently selected from the group consisting of fluoro, chloro, bromo, methyl, ethyl, propyl, isopropyl, methoxy, ethoxy, fluoromethyl, and trifluormethyl.

Preferred compounds of Formula I are those wherein R is optionally substituted phenoxyphenyl or optionally substituted benzyloxyphenyl; R₃ and R₄ together with the nitrogen to which they are attached form a piperidinyl, morpholinyl or pyrrolidinyl group; and Y is an optionally substituted C₂₋₆ alkylene chain.

Preferred compounds of Formula I are also those wherein R₁ is optionally substituted phenoxyphenyl or optionally substituted benzyloxyphenyl; and R₃ and R₄ are independently hydrogen, alkyl or aryl; and Y is an optionally substituted C₁₄ alkylene chain.

Preferred compounds are those of Formula I wherein R₁ is

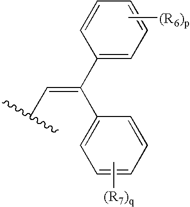

where R₆ and R₇ are independently alkyl, alkoxy, halo, or haloalkyl, and where either or both of p and q are independently greater than 1, R₆ can independently be the same or different, and R₇ can independently be the same or different, and p and q are independently 0-4, preferably 0, 1 or 2. When R₆ and/or R₇ is present, these groups substitute for hydrogen atoms at any available position on the phenyl to which they are attached. Preferred substitution positions are para and meta. Preferably, R₆ and R₇ are independently of C₁₋₄ alkyl, C₁₋₄ alkoxy, halogen, or C₁₋₄ haloalkyl. Useful values of R₆ and R₇ include fluoro, chloro, bromo, methyl, ethyl, propyl, isopropyl, methoxy, ethoxy, fluoromethyl, and trifluormethyl.

Additionally, preferred compounds are those of Formula I wherein R₁ is

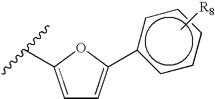

where R₈ is as defined above, and is preferably hydrogen or C₁₋₄ alkyl, such as methyl, ethyl, propyl and isopropyl. R₈ replaces a hydrogen atom at any available position on the phenyl ring.

Additionally, preferred compounds are those of Formula I wherein R₁ is

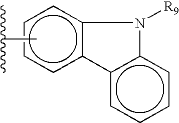

where R₉ is as defined above, and is preferably hydrogen or C₁₋₄ alkyl, such as methyl, ethyl, propyl and isopropyl.

Preferred compounds also are those of Formula I wherein R₁ is naphthyl.

Further, additionally preferred compounds of Formula I are those wherein the —(CH₂)ₙ— is attached to the 3- or 4-position of the phenyl component of the phenoxyphenyl or the benzyloxyphenyl defined by R₁.

Still, additionally preferred compounds of Formula I are those wherein n is 1; Y is C₂₋₆ alkylene; and R₃ and R₄ together with the nitrogen to which they are attached, form a piperidinyl, morpholinyl or pyrrolidinyl group.

Other preferred compounds of Formula I are those wherein n is 1; Y is C₂₋₆ alkylene; and R₃ and R₄ are the same or different and are selected from hydrogen, alkyl, or aryl.

Additionally preferred compounds of Formula I are those wherein R₂ is pyridylalkyl.

Preferred compounds of Formula I are also those wherein R₂ is piperidin-4-ylalkyl, optionally substituted by alkyl, aryl or aralkyl.

The term “alkyl” means a linear or branched C₁₋₁₀ carbon chain, preferably a C₁₋₆ carbon chain. Suitable alkyl groups include, but are not limited to, methyl, ethyl, propyl, isopropyl, butyl, isobutyl, sec-butyl, tert-butyl, 3-pentyl, hexyl and octyl groups.

For purposes of the present invention, the term “alkylene” has the meaning —(CH₂)—, where m is an integer of from 1-6, preferably 2-4. Suitable alkylene chains include but are not limited to methylene, ethylene, propylene, butylene, pentylene and hexylene. The alkylene chain can also be optionally substituted.

The term “optionally substituted,” means optional replacement of one or more carbon-attached hydrogens with halogen, halo(C₁₋₆)alkyl, aryl, heterocycle, cycloalkyl, C₁₋₆ alkyl, C₂₋₆ alkenyl, C₂₋₆ alkynyl, aryl(C₁₋₆)alkyl, aryl(C₂₋₆) alkenyl, aryl(C₂₋₆)alkynyl, cycloalkyl(C₁₋₆)alkyl, heterocyclo(C₁₋₆alkyl), hydroxy(C₁₋₆)alkyl, amino(C₁₋₆)alkyl, carboxy(C₁₋₆)alkyl, alkyloxy(C₁₋₆)alkyl, nitro, amino, ureido, cyano, acylamino, hydroxy, thiol, acyloxy, azido, alkyloxy, carboxy, aminocarbonyl, and C₁₋₆ alkylthiol; and where one or more carbon-attached hydrogens are part of a ring system “optionally substituted,” in addition to those groups listed above, also includes alkyl.

The term “optionally substituted alkylene chain,” means optional replacement of one or more carbon-attached hydrogens with halogen, halo(C₁₋₆)alkyl, aryl, heterocycle, cycloalkyl, C₁₋₆ alkyl, C₂₋₆ alkenyl, C₂₋₆ alkynyl, aryl(C₁₋₆)alkyl, aryl(C₂₋₆)alkenyl, aryl(C₂₋₆)alkynyl, cycloalkyl(C₁₋₆)alkyl, heterocyclo(C₁₋₆)alkyl, hydroxy(C₁₋₆)alkyl, amino(C₁₋₆)alkyl, carboxy(C₁₋₆)alkyl, alkyloxy(C₁₋₆) alkyl, nitro, amino, ureido, cyano, acylamino, hydroxy, thiol, acyloxy, azido, alkyloxy, carboxy, aminocarbonyl, and C₁₋₆ alkylthiol. Preferably, “optionally substituted alkylene chain” will mean replacement with one or more alkyl groups or halogen atoms, preferably alkyl groups.

The term “aryl” means a C₆₋₁₄ mono- or polycyclic aromatic ring system. Suitable carbocyclic aryl groups can be selected from, but are not limited to, phenyl, naphthyl, phenanthryl, anthracyl, indenyl, azulenyl, biphenyl, biphenylenyl and fluorenyl groups. Particularly useful carbocyclic aryl groups are phenyl and naphthyl.

The term “aralkyl” means an alkyl group substituted by a C₆₋₁₄ mono- or polycyclic aromatic ring system. Suitable carbocyclic aryl groups can be selected from, but are not limited to, phenyl, naphthyl, phenanthryl, anthracyl, indenyl, azulenyl, biphenyl, biphenylenyl and fluorenyl groups. Particularly preferred carbocyclic aryl groups are phenyl and naphthyl. Preferred alkyl groups are linear or branched C₁₋₁₀ carbon chain, preferably a C₁₋₆ carbon chain. Suitable alkyl groups include, but are not limited to, methyl, ethyl, propyl, isopropyl, butyl, isobutyl, sec-butyl, tert-butyl, 3-pentyl, hexyl and octyl.

The term “heterocycle” means a 3- to 7-membered monocyclic or a 7-to 14-membered polycyclic non-aromatic ring system, independently containing one or more nitrogen, oxygen or sulfur atoms. Saturated or partially saturated heterocycle groups that are suitable for use in the present invention include, but are not limited to, piperidinyl, tetrahydrofuranyl, pyranyl, piperizinyl, pyrrolidinyl, imidazolindinyl, imidazolinyl, indolinyl, isoindolinyl, quinuclidinyl, morpholinyl, isochromanyl, chromanyl, pyrazolidinyl and pyrazolinyl.

The term “cycloalkyl” means an alkyl group substituted by a 3- to 9-membered monocyclic or a 7- to 14-membered polycyclic non-aromatic carbon ring system. Saturated or partially saturated cycloalkyl groups that are suitable for use in the present invention include, but are not limited to, cyclopropyl, cyclobutyl, cyclopentyl, cyclohexyl, cycloheptyl, cyclooctyl, cyclononyl, cyclopropenyl, cyclobutenyl, cyclopentenyl, cyclohexenyl, cycloheptenyl, cyclooctenyl, cyclononenyl adamantyl, norbomyl, and norbomenyl. Preferred alkyl groups are linear or branched C₁₋₁₀ carbon chain, preferably a C₁₋₆ carbon chain. Suitable alkyl groups include, but are not limited to, methyl, ethyl, propyl, isopropyl, butyl, isobutyl, sec-butyl, tert-butyl, 3-pentyl, hexyl and octyl.

Exemplary compounds that can be employed in this method of invention include, without limitation:

3-(2-piperidinylethyl)-1-(4-(4-flurophenoxy)benzyl) hydantoin;

3-(2-piperidinylethyl)-1-(4-(benzyloxy)benzyl) hydantoin;

3-(2-piperidinylethyl)-1-(3-(4-trifluromethylphenoxy)benzyl) hydantoin;

3-(2-piperidinylethyl)-1-(3-(3,4-dichlorophenoxy)benzyl) hydantoin;

3-(2-piperidinylethyl)-1-(3-(phenoxy)benzyl) hydantoin; and

3-(2-piperidinylethyl)-1-(3-(benzyloxy)benzyl) hydantoin;

as well as pharmaceutically acceptable salts thereof.

Particularly preferred compounds of the invention are selected from:

3-(2-piperidinylethyl)-1-(3-(4-trifluromethylphenoxy)benzyl) hydantoin;

3-(2-piperidinylethyl)-1-(3-(3,4-dichlorophenoxy)benzyl) hydantoin;

3-(2-piperidinylethyl)-1-(3-(benzyloxy)benzyl) hydantoin; and

pharmaceutically acceptable salts thereof.

The invention disclosed herein is meant to encompass all pharmaceutically acceptable salts thereof of the disclosed compounds. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

The invention disclosed herein is also meant to encompass prodrugs of the disclosed compounds. Prodrugs are considered to be any covalently bonded carrier which releases the active parent drug in vivo.

The invention disclosed herein is also meant to encompass the in vivo metabolic products of the disclosed compounds. Such products can result for example from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein is also meant to encompass the disclosed compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O ¹⁷O, ³¹P, ³²P, 35S, ¹⁸F, and ³⁶Cl, respectively.

Some of the compounds disclosed herein can contain one or more asymmetric centers and can thus give rise to enantiomers, diastereomers, and other stereoisomeric forms. The present invention is also meant to encompass all such possible forms as well as their racemic and resolved forms and mixtures thereof. When the compounds described herein contain olefinic double bonds or other centers of geometric asymmetry, and unless specified otherwise, it is intended to include both E and Z geometric isomers. All tautomers are encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule. The phrase “enantiomeric excess” refers to a mixture wherein one enantiomer is present is a greater concentration than its mirror image molecule.

The hydantoins of Formula I can be prepared using methods known to those skilled in the art. Specifically, the hydantoins of the present invention are generally obtained from a method comprising:

(a) reacting an amine protected amino acid with a resin supported hydroxy group to produce a resin supported, amine protected, amino acid;

(b) deprotecting said resin supported amine protected amino acid, to produce a resin supported amino acid having an N-terminus primary amine;

(c) reacting said resin supported amino acid obtained in step (b), with an aldehyde to produce a resin supported enamine;

(d) reducing said resin supported enamine obtained in from step (c), to produce a resin supported amino acid, having an N-terminus secondary amine;

(e) reacting said resin supported amino acid obtained from step (d), with triphosgene, to produce a resin supported amino acid having an N-terminus tertiary amine, wherein said tertiary amine comprises a carbonyl chloride moiety;

(f) reacting said resin supported amino acid obtained from step (e), with a primary amine; and

(g) releasing a product obtained from step (f) from its support, to obtain the compound of Formula I.

For this method, the aldehyde in (c) has the formula:

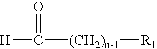

wherein n is 1-3 and R₁ is as defined above.

Additionally, for this method, the primary amine in step (f) is selected from the group consisting of:

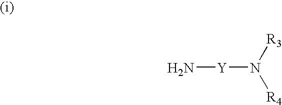

wherein

Y is a C₂₋₆ alkylene; and

R₃ and R₄ are the same or different and are selected from hydrogen, alkyl, or aryl, or R₃ and R₄ together form an alkylene chain having 4 to 5 carbon atoms, optionally substituted with an alkyl or aryl moiety, or said alkylene chain is optionally interrupted by an oxygen atom or —NR₅, where R₅ is hydrogen or alkyl;

(i) pyridylalkyl amine; and

(ii) an optionally substituted piperidin-4-ylalkyl amine, wherein optional substituents are selected from the group consisting of alkyl, aryl or aralkyl.

The general method of making hydantoins of the present invention is shown in the following reaction scheme.

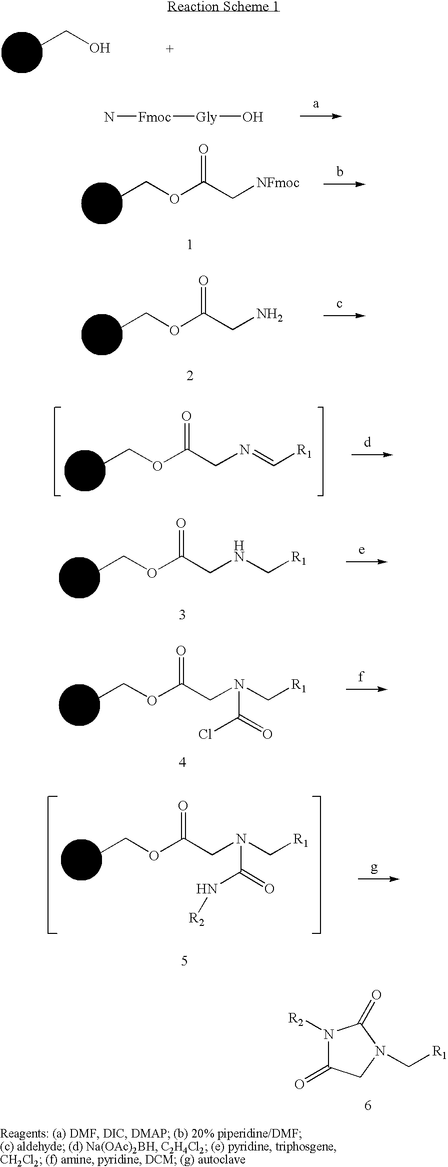

Other commercially available protected amino acids can be substituted for N-Fmoc-Gly-OH (i.e., FMOC-NH-Gly-COOH) in step 1 to form compounds of Formula I where R is other than hydrogen.

Compounds of Formula I, wherein n is 0, can also be prepared according to a modification of Scheme I above, comprising:

(a) reacting a resin supported hydroxymethyl group with halogenated acetic acid to produce a supported halogenated acetate;

(b) reacting said supported halogenated acetate from step (a) with an R₁-containing primary amine, to form a supported R₁-substituted glycine;

(c) reacting said supported R₁-substituted glycine obtained from step (b), with triphosgene, to produce a resin supported amino acid having an N-terminus tertiary amine, wherein said tertiary amine comprises a carbonyl chloride moiety;

(d) reacting said resin supported amino acid obtained from step (c), with a primary amine; and

(e) releasing a product obtained from step (d) from its support, to obtain the compound of Formula I wherein n is 0.

The general method of making hydantoins of Formula I, where n is 0, is shown in reaction Scheme 2.

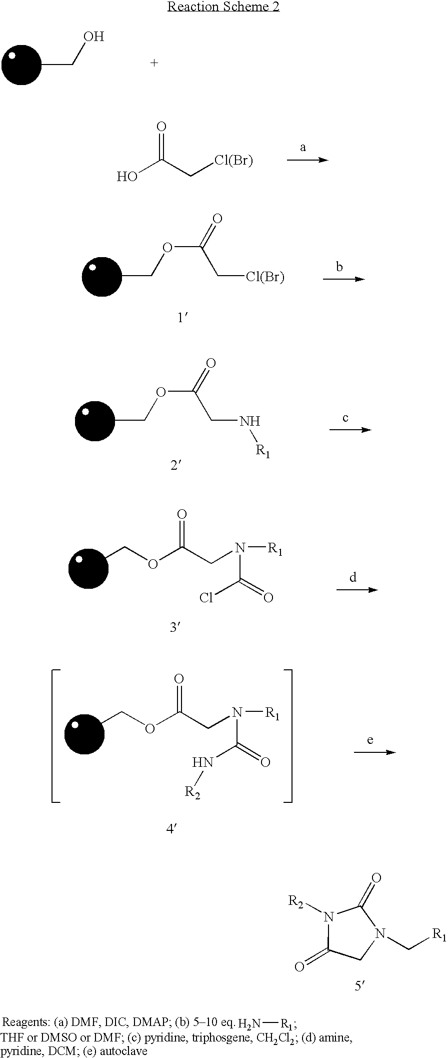

Those of ordinary skill in the art would be readily aware of corresponding solution phase methods of making the compound of Formula I.

The invention is also directed to a method for treating disorders responsive to the blockade of sodium channels in mammals suffering therefrom. The hydantoin compounds of the invention can be used to treat humans or companion animals, such as dogs and cats. Particular preferred embodiments of the hydantoins of the invention for use in treating such disorders are represented as previously defined for Formula I.

The compounds of the present invention are assessed by electrophysiological assays in dissociated hippocampal neurons for sodium channel blocker activity. These compounds also can be assayed for binding to the neuronal voltage-dependent sodium channel using rat forebrain membranes and [³H]BTX-B.

Sodium channels are large transmembrane proteins that are expressed in various tissues. They are voltage sensitive channels and are responsible for the rapid increase of Na⁺ permeability in response to depolarization associated with the action potential in many excitable cells including muscle, nerve and cardiac cells.

One aspect of the present invention is the discovery of the mechanism of action of the compounds herein described as specific Na⁺ channel blockers. Based upon the discovery of this mechanism, these compounds are contemplated to be useful in treating or preventing neuronal loss due to focal or global ischemia, and in treating or preventing neurodegenerative disorders including ALS, anxiety, and epilepsy. They are also expected to be effective in treating, preventing or ameliorating neuropathic pain, surgical pain, chronic pain and tinnitus. The compounds are also expected to be useful as antiarrhythmics, anesthetics and antimanic depressants.

The present invention is directed to compounds of Formula I that are blockers of voltage-sensitive sodium channels. According to the present invention, those compounds having preferred sodium channel blocking properties exhibit an IC₅₀ of about 100 μM or less in the electrophysiological assay described herein. Preferably, the compounds of the present invention exhibit an IC₅₀ of 10 μM or less. Most preferably, the compounds of the present invention exhibit an IC₅₀ of about 1.0 μM or less. Compounds of the present invention can be tested for their Na+channel blocking activity by the following binding and electrophysiological assays.

In Vitro Binding Assay:

The ability of compounds of the present invention to modulate either site 1 or site 2 of the Na+channel was determined following the procedures fully described in Yasushi, J. Biol. Chem. 261:6149-6152 (1986) and Creveling, Mol. Pharmacol. 23:350-358 (1983), respectively. Rat forebrain membranes are used as sources of Na⁺ channel proteins. The binding assays are conducted in 130 μM choline chloride at 37° C. for 60-minute incubation with [³H] saxitoxin and [³H] batrachotoxin as radioligands for site 1 and site 2, respectively.

In Vivo Pharmacology:

The compounds of the present invention can be tested for in vivo anticonvulsant activity after i.v., p.o. or i.p. injection using a number of anticonvulsant tests in mice, including the maximum electroshock seizure test (MES). Maximum electroshock seizures are induced in male NSA mice weighing between 15-20 g and male Sprague-Dawley rats weighing between 200-225 g by application of current (50 mA, 60 pulses/sec, 0.8 msec pulse width, 1 sec duration, D.C., mice; 99 mA, 125 pulses/sec, 0.8 msec pulse width, 2 sec duration, D.C., rats) using a Ugo Basile ECT device (Model 7801). Mice are restrained by gripping the loose skin on their dorsal surface and saline-coated corneal electrodes were held lightly against the two corneae. Rats are allowed free movement on the bench top and ear-clip electrodes are used. Current is applied and animals are observed for a period of up to 30 seconds for the occurrence of a tonic hindlimb extensor response. A tonic seizure is defined as a hindlimb extension in excess of 90 degrees from the plane of the body. Results are treated in a quantal manner.

The compounds can be tested for their antinociceptive activity in the formalin model as described in Hunskaar, S., O. B. Fasmer, and K. Hole, J. Neurosci. Methods 14: 69-76 (1985). Male Swiss Webster NIH mice (20-30 g; Harlan, San Diego, Calif.) are used in all experiments. Food is withdrawn on the day of experiment. Mice are placed in Plexiglass jars for at least 1 hour to accommodate to the environment. Following the accommodation period mice are weighed and given either the compound of interest administered i.p. or p.o., or the appropriate volume of vehicle (10% Tween-80). Fifteen minutes after the i.p. dosing, and 30 minutes after the p.o. dosing mice are injected with formalin (20 μL of 5% formaldehyde solution in saline) into the dorsal surface of the right hind paw. Mice are transferred to the Plexiglass jars and monitored for the amount of time spent licking or biting the injected paw. Periods of licking and biting are recorded in 5 minute intervals for 1 hour after the formalin injection. All experiments are done in a blinded manner during the light cycle. The early phase of the formalin response is measured as licking/biting between 0-5 minutes, and the late phase is measured from 15-50 minutes. Differences between vehicle and drug treated groups are analyzed by one-way analysis of variance (ANOVA). A P value <0.05 is considered significant. Activity in blocking the acute and second phase of formalin-induced paw-licking activity is indicative that compounds are considered to be efficacious for acute and chronic pain.

The compounds can be tested for their potential for the treatment of chronic pain (antiallodynic and antihyperalgesic activities) in the Chung model of peripheral neuropathy. Male Sprague-Dawley rats weighing between 200-225 g are anesthetized with halothane (1-3% in a mixture of 70% air and 30% oxygen) and their body temperature is controlled during anesthesia through use of a homeothermic blanket. A 2-cm dorsal midline incision is then made at the L5 and L6 level and the para-vertibral muscle groups retracted bilaterally. L5 and L6 spinal nerves are then be exposed, isolated, and tightly ligated with 6-0 silk suture. A sham operation is performed exposing the contralateral L5 and L6 spinal nerves as a negative control.

Tactile Allodynia: Rats are transferred to an elevated testing cage with a wire mesh floor and allowed to acclimate for five to ten minutes. A series of Semmes-Weinstein monofilaments are applied to the plantar surface of the hindpaw to determine the animal's withdrawal threshold. The first filament used possesses a buckling weight of 9.1 gms (0.96 log value) and is applied up to five times to see if it elicited a withdrawal response. If the animal has a withdrawal response then the next lightest filament in the series is applied up to five times to determine if it can elicit a response. This procedure is repeated with subsequent less filaments until there is no response and the lightest filament that elicits a response is recorded. If the animal does not have a withdrawal response from the initial 9.1 gms filament then subsequent filaments of increased weight are applied until a filament elicits a response and this filament is then recorded. For each animal, three measurements are made at every time point to produce an average withdrawal threshold determination. Tests are performed prior to and at 1, 2, 4 and 24 hours post drug administration. Tactile allodynia and mechanical hyperalgesia tests were conducted concurrently.

Mechanical Hyperalgesia: Rats are transferred to an elevated testing cage with a wire mesh floor and allowed to acclimate for five to ten minutes. A slightly blunted needle is touched to the plantar surface of the hindpaw causing a dimpling of the skin without penetrating the skin. Administration of the needle to control paws typically produces a quick flinching reaction, too short to be timed with a stopwatch and arbitrarily gives a withdrawal time of 0.5 second. The operated side paw of neuropathic animals exhibits an exaggerated withdrawal response to the blunted needle. A maximum withdrawal time of ten seconds is used as a cutoff time. Withdrawal times for both paws of the animals are measured three times at each time point with a five-minute recovery period between applications. The three measures are used to generate an average withdrawal time for each time point. Tactile allodynia and mechanical hyperalgesia tests are conducted concurrently.

The compounds can be tested for their neuroprotective activity after focal and global ischemia produced in rats or gerbils according to the procedures described in Buchan et al. (Stroke, Suppl. 148-152 (1993)) and Sheardown et al. (Eur. J. Pharmacol. 236:347-353 (1993)) and Graham et al. (J. Pharmacol. Exp. Therap. 276:1-4 (1996)).

The compounds can be tested for their neuroprotective activity after traumatic spinal cord injury according to the procedures described in Wrathall et al. (Exp. Neurology 137:119-126 (1996)) and Iwasaki et al. (J. Neuro Sci. 134:21-25 (1995)).

Electrophysiological Assay:

An electrophysiological assay was used to measure potencies of compounds of the present invention as antagonists of rBIIa/beta 1 sodium channels expressed in Xenopus oocytes.

Preparation of cRNA encoding cloned rat brain type IIa (rBIIa) and beta 1 (β1): cDNA clones encoding the rat brain beta 1 subunit are cloned in house using standard methods, and mRNA are prepared by standard methods. MRNA encoding rBIIa is provided by Dr. A. Golden (UC Irvine). The mRNAs are diluted and stored at −80° C. in 1 μL aliquots until injection.

Preparation of oocytes: Mature female Xenopus laevis are anaesthetized (20-40 min) using 0.15% 3-aminobenzoic acid ethyl ester (MS-222) following established procedures (Woodward, R. M., et al., Mol. Pharmacol. 41:89-103 (1992)).

Two to six ovarian lobes are surgically removed. Oocytes at developmental stages V-VI are dissected from the ovary, wherein the oocytes are still surrounded by enveloping ovarian tissues. Oocytes are defolliculated on the day of surgery by treatment with collagenase (0.5 mg/mL Sigma Type I, or Boehringer Mannheim Type A, for 0.5-1 hr). Treated oocytes are vortexed to dislodge epithelia, washed repeatedly and stored in Barth's medium containing 88 mM NaCl, 1 mM KCl, 0.41 mM CaCl₂, 0.33 mM Ca(NO₃)₂, 0.82 mM MgSO₄, 2.4 mM NaHCO₃, 5 mM HEPES, pH 7.4 adjusted with 0.1 mg/mL gentamycin sulphate.

Micro-injection of oocytes: Defolliculated oocytes are micro-injected using a Nanoject injection system (Drummond Scientific Co., Broomall, Pa.). Injection pipettes are beveled to minimize clogging. Tip diameter of injection pipettes is 15-35 μm. Oocytes are microinjected with approximately 50 nL 1:10 ratio mixtures of cRNAs for rBIIa and beta 1 respectively.

Electrophysiology: Membrane current responses are recorded in frog Ringer solution containing 115 mM NaCl, 2 mM KCl, 1.8 mM CaCl₂, 5 mM HEPES, pH 7.4. Electrical recordings are made using a conventional two-electrode voltage clamp (Dagan TEV-200) over periods ranging between 1-7 days following injection. The recording chamber is a simple gravity fed flow-through chamber (volume 100-500 mL depending on adjustment of aspirator). Oocytes are placed in the recording chamber, impaled with electrodes and continuously perfused (5-15 mL min⁻¹) with frog Ringer's solution. The tested compounds are applied by bath perfusion.

Voltage protocols for evoking sodium channel currents: The standard holding potential for whole oocyte clamp is −120 mV. Standard current-voltage relationships are elicited by 40 ms depolarizing steps starting from −60 mV to +50 mV in 10 mV increments. Peak currents are measured as the maximum negative current after depolarizing voltage steps. The voltage from maximum current response is noted and used for the next voltage protocol.

The purpose is to find compounds that are state dependent modifiers of neuronal sodium channels. Preferably, the compounds have a low affinity for the rested/closed state of the channel, but a high affinity for the inactivated state. The following voltage protocol is used to measure a compounds affinity for the inactivated state. Oocytes are held at a holding potential of −120 mV. At this membrane voltage, nearly all of the channels are in the closed state. Then a 4 second depolarization is made to the voltage where the maximum current is elicited. At the end of this depolarization, nearly all the channels are in the inactivated state. A 10 ms hyperpolarizing step is then made in order to remove some channels from the inactivated state. A final depolarizing test pulse is used to assay the sodium current after this prolonged depolarization (see analysis below). Sodium currents are measured at this test pulse before and after the application of the tested compound. Data is acquired using PCLAMP 8.0 software and analyzed with CLAMPFIT software (Axon instruments).

Data analysis: Apparent inhibition constants (K; values) for antagonists are determined from single point inhibition data using the following equation (a generalized form of the Cheng-Prusoff equation) (Leff, P. and I. G. Dougall, TiPS 14:110-112 (1993)).

Kᵢ=(FR/1−FR)*[drug] Eq. 1

Where FR is the fractional response and is defined as sodium current elicited from the final depolarizing test pulse prior to application of the drug divided by the sodium current measured in the presence of the drug. [drug] is the concentration of the drug used.

Drugs: Drugs are initially made up at concentrations of 2-10 mM in DMSO. Dilutions are then made to generate a series of DMSO stocks over the range 0.3 μM to 10 mM—depending upon the potency of the compound. Working solutions are made by 1000-3000 fold dilution of stocks into Ringer. At these dilutions DMSO alone has little or no measurable effects on membrane current responses. DMSO stocks of drugs are stored in the dark at 4° C. Ringer solutions of drugs are made up fresh each day of use.

Compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds can be administered to mammals, e.g. humans, orally at a dose of 0.0025 to 50 mg/kg, or an equivalent amount of the pharmaceutically acceptable salt thereof, per day of the body weight of the mammal being treated for epilepsy, neurodegenerative diseases, anesthetic, arrhythmia, manic depression, and chronic pain. For intramuscular injection, the dose is generally about one-half of the oral dose.

In the method of treatment or prevention of neuronal loss in global and focal ischemia, brain and spinal cord trauma, hypoxia, hypoglycemia, status epilepsy and surgery, the compound can be administrated by intravenous injection at a dose of about 0.025 to about 10 mg/kg.

The unit oral dose can comprise from about 0.01 to about 50 mg, preferably about 0.1 to about 10 mg of the compound. The unit dose can be administered one or more times daily as one or more tablets each containing from about 0.1 to about 10, conveniently about 0.25 to 50 mg of the compound or its solvates.

In addition to administering the compound as a raw chemical, the compounds of the invention can be administered as part of a pharmaceutical preparation containing suitable pharmaceutically acceptable carriers comprising excipients and auxiliaries, which facilitate processing of the compounds into preparations that can be used pharmaceutically. Preferably, the preparations, particularly those preparations which can be administered orally and which can be used for the preferred type of administration, such as tablets, dragees, and capsules, and also preparations which can be administered rectally, such as suppositories, as well as suitable solutions for administration by injection or orally, contain from about 0.01 to 99 percent, preferably from about 0.25 to 75 percent of active compound(s), together with the excipient.

Also included within the scope of the present invention are the non-toxic pharmaceutically acceptable salts of the compounds of the present invention. Acid addition salts are formed by mixing a solution of the particular hydantoins of the present invention, with a solution of a pharmaceutically acceptable non-toxic acid such as, but not limited to: acetic acid, benzoic acid, carbonic acid, citric acid, dichloroacetic acid, dodecylsulfonic acid, 2-ethylsuccinic acid, fumaric acid, glubionic acid, gluconic acid, hydrobromic acid, hydrochloric acid, 3-hydroxynaphthoic acid, isethionic acid, lactic acid, lactobionic acid, levulinic acid, maleic acid, malic acid, malonic acid, methanesulfic acid, methanesulfonic acid, nitric acid, oxalic acid, phosphoric acid, propionic acid, sulfuric acid, sulfamic acid, saccharic acid, succinic acid, tartaric acid, and the like. Basic amine salts are formed by mixing a solution of the hydantoin compounds of the present invention with a solution of a pharmaceutically acceptable non-toxic acid, such as those listed above, preferably, hydrochloric acid or carbonic acid.

The pharmaceutical compositions of the invention can be administered to any animal that may experience the beneficial effects of the compounds of the invention. Foremost among such animals are mammals, e.g., humans and companion animals such as, dogs and cats, although the invention is not intended to be so limited.

The pharmaceutical compositions of the present invention can be administered by any means that achieve their intended purpose. For example, administration can be by parenteral, subcutaneous, intravenous, intramuscular, intraperitoneal, transdermal, or buccal routes. Alternatively, or concurrently, administration can be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions can be used, which can optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers can be added.

Possible pharmaceutical preparations, which can be used rectally, include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances which increase the viscosity of the suspension, and include, for example, sodium carboxymethyl cellulose, sorbitol, and/or dextran. Optionally, the suspension can also contain stabilizers.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

Preparation of Hydantoins by Parallel Synthesis

Wang resin (i.e., 4-benzyloxybenzyl alcohol polystyrene) (6 g, 5.34 mmol) is placed in 250 mL of peptide vessel. Dimethylformamide (DMF) (70 mL) and F-moc glycine (9.6 g, 32.4 mmol) are added, followed by dicarbazine (DIC) (6 equiv.) and dimethylaminopyridine (DMAP) (0.5 equiv.). The reaction vessel is secured on a shaker table and allowed to agitate overnight. The resin is then washed with DMF (6×70 mL), MeOH (4×70 mL), and dichloromethane (i.e., DCM) (6×70 mL) and the solvent is removed under reduced pressure to give resin 1.

After removal of the Fmoc protecting group with 20% piperidine in DMF, the free amine 2 is reacted with an appropriate aldehyde (e.g., 4-(4-fluorophenoxy benzaldehyde) in dichloroethane (DCE) and in the presence of Na(OAc)₃BH (8 equiv.) thereby resulting in the formation of resin bound compound 3. The resin is then washed with H₂O (1×70 mL), DMF (6×70 mL), MeOH (4×70 mL), and DMC (6×70 mL). The resin is then dried under vacuum overnight.

Resin 3 (300 g, 0.27 mmol) is then treated with a 0.18 M solution of triphosgene (3 equiv.) in DCM, in the presence of pyridine, resulting in the formation of carbonyl chloride compound 4. Compound 4 is agitated for about 3 hours and washed with DCM. Compound 4 is then suspended in a 0.5 M solution of pyridine (10 equiv.), then treated with an appropriate amine (e.g., 1-(2-aminoethyl)-piperidine) (10 equiv.) and agitated for about 15 hours. Upon release from the resin support, the released compound undergoes ring closure to form the hydantoin product 6, in solution. The solvent is removed from the hydantoin product and the product is washed with DCM and filtered. The resulting filtrate is evaporated and the hydantoin product 6 is recovered in solid form. The recovered product is purified by flash column chromatography.

The compounds listed in Table 1 were prepared according to this parallel synthesis. These compounds were tested in the electrophysiological assay described above and apparent inhibition constants (expressed as Kᵢ values) were determined. The compounds listed in Table 1 have Kᵢ values of between 290 nM and 980 nM. The data demonstrates that compounds of the invention are potent blockers of the sodium channel.

TABLE 1
REPRESENTATIVE HYDANTOIN COMPOUNDS OF THE INVENTION

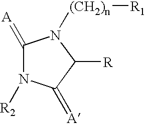

| n | A | A′ | R | R₁ | R₂ | NMR Data |
|---|---|---|---|---|---|---|
| 1 | O | O | H | 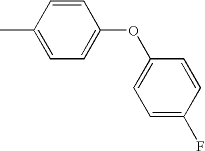 | 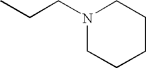 | ¹H NMR (400 MHz, CD₃OD): δ 1.32 (s, 1H), 1.45 (bs, 2H), 1.55 (bs, 4H), 2.43-2.55 (bd, 5H), 3.72 (m, 2H), 3.77 (s, 2H), 4.50 (s, 2H), 6.99 (m, 6H), 7.15 (t, 2H). |
| 1 | O | O | H | 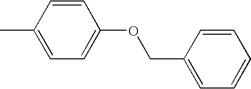 | 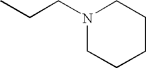 | ¹H NMR (400 MHz, CD₃OD): δ 1.31 (bs, 2H), 1.55 (bs, 4H), 2.45 (bs, 4H), 2.55 (t, 2H), 3.72 (m, 4H), 4.57 (s, 2H), 5.05 (s, 2H), 6.99 (t, 2H), 7.11 (t, 2H), 7.40 (m, 5H). |
| 1 | O | O | H | 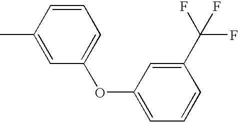 | 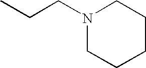 | ¹H NMR (400 MHz, CD₃OD): δ 1.33 (bs, 2H), 1.57 (bs, 4H), 2.35 (bs, 4H), 2.56 (bd, 2H), 3.65 (t, 2H), 3.79 (s, 2H), 4.58(s, 2H), 6.99 (t, 2H), 7.05 (t, 1H), 7.11 (t, 1H), 7.21 (t, 1H), 7.41 (m, 2H), 7.51 (m, 1H). |
| 1 | O | O | H | 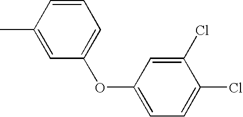 | 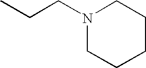 | ¹H NMR (400 MHz, CD₃OD): δ 1.32 (bd, 2H), 1.45 (bt, 4H), 2.41 (bs, 4H), 2.51 (bt, 2H), 3.67 (m, 2H), 3.77 (s, 2H) , 4.60 (s, 2H), 6.85 (m, 1H), 6.99 (m, 2H), 7.15 (m, 2H), 7.41 (bm, 2H). |
| 1 | O | O | H | 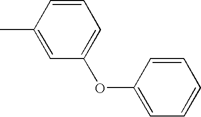 | 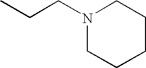 | ¹H NMR (400 MHz, CD₃OD): δ 1.29 (bd, 2H), 1.51 (bt, 4H), 2.39-2.61 (bd, 6H), 3.62 (t, 2H), 3.72 (s, 2H), 4.51 (s, 2H), 6.99-7.07 (m, 5H), 7.12 (t, 1H), 7.45 (m, 3H). |
| 1 | O | O | H | 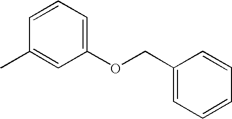 | 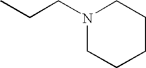 | ¹H NMR (400 MHz, CD₃OD): δ 1.32 (bs, 2H), 1.51 (bs, 4H), 2.41-2.51 (bd, 5H), 3.72 (t, 4H), 4.67 (s, 2H), 5.11 (s, 2H) , 6.88 (t, 3H), 6.95 (m, 2H), 6.99 (m, 1H), 7.25-7.48 (m, 6H). |

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

What is claimed is:

1. A compound having the Formula I: 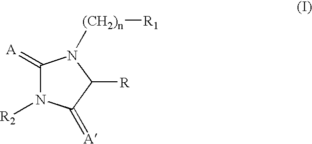
or a pharmaceutically acceptable salt, or solvate thereof, wherein:
n is 0 to 3;
A and A′ are independently oxygen or sulfur;
R is hydrogen, linear or branched alkyl, benzyl, hydroxybenzyl, thioalkyl, alkylthioalkyl, hydroxyalkyl, aminoalkyl, guanidinylalkyl, carboxyalkyl or aminocarboxyalkyl;
R₁ is selected from the group consisting of:
(i) optionally substituted phenoxyphenyl;
(ii) optionally substituted benzyloxyphenyl;
(iii) optionally substituted phenylthiophenyl;
(iv) optionally substituted benzylthiophenyl;
(v) optionally substituted phenylaminophenyl;
(vi) optionally substituted benzylaminophenyl; and
(vii) 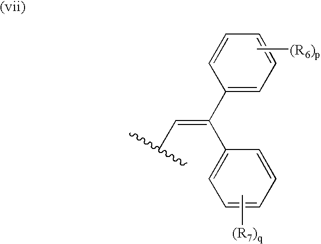
wherein each occurrence of R₆ and each occurrence of R₇ are independently hydrogen or alkyl; and
p and q are independently integers from zero to 4; 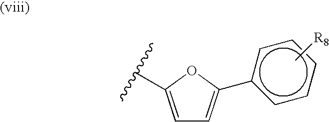
wherein R₈ is hydrogen, halogen, hydroxy, C₁₋₆ alkyl, C₁₋₆ alkoxy, cyano, amino, or nitro; 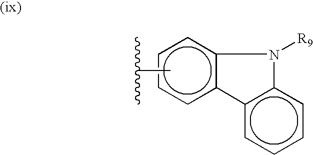
wherein R₉ is hydrogen or C₁₋₆ alkyl; and
(x) optionally substituted naphthdyl; and
R₂ is selected from the group consisting of: 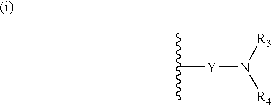
where
Y is an optionally substituted C₂₋₆ alkylene, and
R₃ and R₄ are the same or different and are hydrogen, alkyl, or aryl, or R₃ and R₄ together form an alkylene chain having 3 to 5 carbon atoms, optionally substituted with an alkyl or aryl moiety, or said alkylene chain is optionally interrupted by an oxygen atom or —NR₅, where R₅ is hydrogen or C₁₋₆ alkyl;
(ii) pyridylalkyl; and
(iii) piperidin-4-ylalkyl, optionally substituted by alkyl, aryl or aralkyl.

2. The compound according to claim 1, wherein R₂ is —YNR₃R₄, and an optionally substituted C₂₋₆ alkylene.

3. The compound according to claim 2, wherein:
R₁ is optionally substituted phenoxyphenyl or optionally substituted benzyloxyphenyl; R₃ and R₄ together form an alkylene chain having 4 to 5 carbon atoms; and Y is an optionally substituted C₂₋₄ alkylene chain.

4. The compound according to claim 3, wherein R₃ and R₄ together form an alkylene chain of 5 carbon atoms; and Y is an optionally substituted C₂₋₄ alkylene chain.

5. The compound according to claim 3, wherein R₃ and R₄ together form an alkylene chain of 4 carbon atoms, and Y is an optionally substituted C₂₋₄ alkylene chain.

6. The compound according to claim 2, wherein Y is ethylene or propylene.

7. The compound according to claim 2, wherein:
R¹ is optionally substituted phenoxyphenyl or optionally substituted benzyloxyphenyl; R₃ and R₄ are independently hydrogen, alkyl or aryl; and Y is an optionally substituted C₂₋₄ alkylene chain.

8. The compound according to claim 1, wherein n is 1.

9. The compound according to claim 1, wherein n is 0.

10. The compound according to claim 1, wherein R₁ is an optionally substituted phenoxyphenyl.

11. The compound according to claim 1, wherein R₁ is an optionally substituted benzyloxyphenyl.

12. The compound according to claim 1, wherein —(CH₂)ₙ— is attached to the 3- or 4-position of the phenyl component of the phenoxyphenyl or the benzyloxyphenyl defined by R₁.

13. The compound according to claim 10, wherein n is 1; R₂ is —YNR₃R₄, Y is C₂₋₆ alkylene; and R₃ and R₄ together form an alkylene chain having 4 to 5 carbon atoms.

14. The compound according to claim 11, wherein n is 1; R₂ is —YNR₃R₄, Y is C₂₋₆ alkylene; and R₃ and R₄ together form an alkylene chain having 4 to 5 carbon atoms.

15. The compound according to claim 10, wherein n is 1; R₂ is —YNR₃R₄, Y is C₂₋₆ alkylene; and R₃ and R₄ are the same or different and are selected from hydrogen, alkyl, or aryl.

16. The compound according to claim 11, wherein n is 1; R₂ is —YNR₃R₄, Y is C₂₋₆ alkylene; and R₃ and R₄ are the same or different and are selected from hydrogen, alkyl, or aryl.

17. The compound according to claim 1, wherein R₁ is

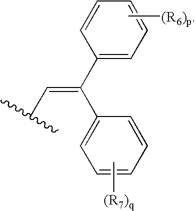

18. The compound according to claim 17, wherein R₂ is

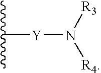

19. The compound according to claim 17 wherein Y is a C₂₋₆ alkylene, and R₃ and R₄ are the same or different and are selected from hydrogen, alkyl, or aryl.

20. The compound according to claim 18, wherein Y is a C₂₋₆ alkylene, and, R₃ and R₄ together form an alkylene chain having 4 to 5 carbon atoms, which is optionally substituted with an alkyl or aryl moiety.

21. The compound according to claim 20, wherein said alkylene chain formed from taking R₃ and F₄ together is further interrupted by an oxygen atom or —NR₅, where R₅ is hydrogen or C₁₋₆ alkyl.

22. The compound according to claim 1, wherein R₁ is

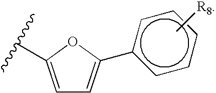

23. The compound according to claim 22, wherein R₂ is

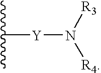

24. The compound according to claim 23, wherein Y is a C₂₋₆ alkylene, and R₃ and R₄ are the same or different and are selected from hydrogen, alkyl, or aryl.

25. The compound according to claim 23, wherein Y is a C₂₋₆ alkylene, and, R₃ and R₄ together form an alkylene chain having 4 to 5 carbon atoms, which is optionally substituted with an alkyl or aryl moiety.

26. The compound according to claim 25, wherein said alkylene chain formed from taking R₃ and R₄ together is further interrupted by an oxygen atom or —NR₅, where R₅ is hydrogen or alkyl.

27. The compound according to claim 1, wherein R₁ is

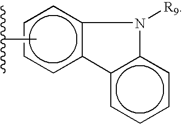

28. The compound according to claim 27, wherein R₂ is

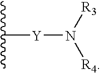

29. The compound according to claim 28, wherein Y is a C₂₋₆ alkylene, and R₃ and R₄ are the same or different and are selected from hydrogen, alkyl, or aryl.

30. The compound according to claim 28, wherein Y is a C₂₋₆ alkylene, and, R₃ and R₄ together form an alkylene chain having 4 to 5 carbon atoms, optionally substituted with an alkyl or aryl moiety.

31. The compound according to claim 30, wherein said alkylene chain formed from taking R₃ and R₄ together is further interrupted by an oxygen atom or —NR₅, where R₅ is hydrogen or alkyl.

32. The compound according to claim 1, wherein R₁ is naphthyl.

33. The compound according to claim 31, wherein R₂ is

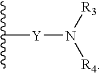

34. The compound according to claim 32, wherein Y is a C₂₋₆ alkylene, and R₃ and R₄ are the same or different and are selected from hydrogen, alkyl, or aryl.

35. The compound according to claim 33, wherein Y is a C₂₋₆ alkylene, and, R₃ and R₄ together form an alkylene chain having 4 to 5 carbon atoms, which is optionally substituted with an alkyl or aryl moiety.

36. The compound according to claim 35, wherein said alkylene chain is further interrupted by an oxygen atom or —NR₅, where R₅ is hydrogen or alkyl.

37. The compound according to claim 1, wherein said compound is selected from the group consisting of:
3-(2-piperidinylethyl)-1-(4-(4-flurophenoxy)benzyl) hydantoin;
3-(2-piperidinylethyl)-1-(4-(benzyloxy)benzyl) hydantoin;
3-(2-piperidinylethyl)-1-(3-(4-trifluromethylphenoxy)benzyl) hydantoin;
3-(2-piperidinylethyl)-1-(3-(3,4-dichlorophenoxy)benzyl) hydantoin;
3-(2-piperidinylethyl)-1-(3-(phenoxy)benzyl) hydantoin; and
3-(2-piperidinylethyl)-1-(3-(benzyloxy)benzyl) hydantoin.

38. A pharmaceutical composition, comprising the compound of claim 1, and a pharmaceutically acceptable carrier or diluent.

39. A method of making a compound according to claim 1 wherein said method comprises:
(a) reacting an amine-protected amino acid with a resin-supported hydroxy group to produce a resin-supported, amine-protected, amino acid;
(b) deprotecting said resin-supported amine-protected amino acid, to produce a resin-supported amino acid having an N-terminus primary amine;
(c) reacting said resin-supported amino acid obtained in step (b), with an aldehyde to produce a resin-supported enamine;
(d) reducing said resin-supported enamine obtained in from step (c), to produce a resin-supported amino acid, having an N-terminus secondary amine;
(e) reacting said resin-supported amino acid obtained from step (d), with triphosgene, to produce a resin-supported amino acid having an N-terminus tertiary amine, wherein said tertiary amine comprises a carbonyl chloride moiety;
(f) reacting said resin-supported amino acid obtained from step (e), with a primary amine; and
(g) releasing a product obtained from step (f) from its support, to obtain the compound of Formula I.

40. The method according to claim 39, wherein said resin is a Wang resin.

41. The method according to claim 39, wherein step (a) is carried out in the presence of DMF, DIC and DMAP.

42. The method according to claim 39, wherein step (b) is carried out in the presence of piperidine and DMF.

43. The method according to claim 39, wherein said aldehyde of step (c) has the formula: 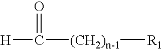
wherein:
n is an integer from 1-3; and
R₁ is selected from the group consisting of:
(i) optionally substituted phenoxyphenyl;
(ii) optionally substituted benzyloxyphenyl;
(iii) optionally substituted phenylthiophenyl;
(iv) optionally substituted benzylthiophenyl;
(v) optionally substituted phenylaminophenyl;
(vi) optionally substituted benzylaminophenyl; 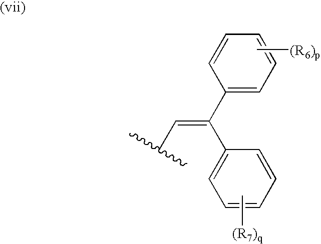
wherein R₆ and R₇ are independently hydrogen or alkyl; and p and q are independently integers from 0 to 4; 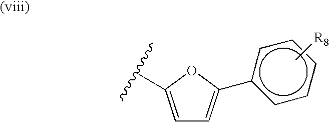
wherein R₈ is hydrogen or alkyl, 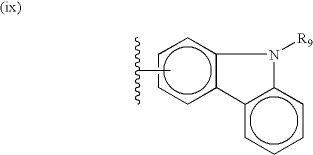
wherein R₉ is hydrogen or alkyl; and
(x) naphthyl.

44. The method according to claim 39, wherein said primary amine of step (f) is selected from the group consisting of:
(i) an amine of the formula: 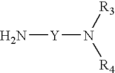
wherein
Y is an optionally substituted C₂₋₆ alkylene; and
R₃ and R₄ are the same or different and are selected from hydrogen, alkyl, or aryl, or R₃ and R₄ together form an alkylene chain having 4 to 5 carbon atoms, optionally substituted with an alkyl or aryl moiety, and said alkylene chain is optionally interrupted by an oxygen atom or —NR₅, where R₅ is hydrogen or alkyl;
(ii) pyridylalkyl amine; and
(iii) an optionally substituted piperidin-4-ylalkyl amine, wherein optional substituents are selected from the group consisting of alkyl, aryl or aralkyl.

45. The method according to claim 44, wherein said primary amine is 1-(2-aminoethyl)piperidine; (2-aminoethyl)pyrrolidine; or di(2-propyl)(2-aminoethyl) amine.

46. The method according to claim 39, wherein step (e) is carried out in the presence of DMF and pyridine.

47. A method of treating, preventing or ameliorating a disorder responsive to blockage of sodium channels in a mammal, comprising administering to a mammal in need thereof an effective amount of a compound according to claim 1, or pharmaceutically acceptable salt thereof.

48. The method according to claim 47, wherein said disorder is selected from the group consisting of: neuronal damage; a neurodegenerative condition, acute or chronic pain, depression, and diabetic neuropathy.

49. The method according to. Claim 47, wherein said neuronal damage is caused by focal or global ischemia.

50. The method according to claim 47, wherein said neurodegenerative condition is amyotrophic lateral sclerosis (ALS).

51. The compound according to claim 1, wherein said compound functions as an antitinnitus agent, anticonvulsant, antiarrhythmic, local anesthetic, or antimanic depressant.

52. A method of treating a mammal suffering from a disorder responsive to blockage of sodium channels, said method comprising administering to said mammal a compound according to claim 1, or pharmaceutically acceptable salt thereof, in an amount that is effective for treating said disorder.

53. The method according to claim 52 wherein said mammal is a human, dog or cat.

54. The method according to claim 53, wherein said disorder is selected from the group consisting of: neuronal damage; a neurodegenerative condition, acute or chronic pain, depression, and diabetic neuropathy.

55. The method according to claim 53, wherein said neuronal damage is caused by focal or global ischemia.

56. The method according to claim 53, wherein said neurodegenerative condition is amyotrophic lateral sclerosis (ALS).

57. The compound according to claim 37, wherein the compound is selected from the group consisting of:
3-(2-piperidinylethyl)-1-(3-(4-trifluromethylphenoxy)benzyl) hydantoin;
3-(2-piperidinylethyl)-1-(3-(3,4-dichlorophenoxy)benzyl)hydantoin; and
3-(2-piperidinylethyl)-1-(3-(benzyloxy)benzyl)hydantoin.
